(19) United States
(12) Patent Application Publication
(10) Pub. No.: US 2024/0302264 A1
(43) Pub. Date: Sep. 12, 2024

(54) METHODS OF ANALYZING SHAPED PARTICLES CONTAINING CELLS USING FLUORESCENCE ACTIVATED CELL SORTING

(71) Applicant: THE REGENTS OF THE UNIVERSITY OF CALIFORNIA, Oakland, CA (US)

(72) Inventors: Dino Di Carlo, Los Angeles, CA (US); Joseph de Rutte, Los Angeles, CA (US); Robert Dimatteo, Sherman Oaks, CA (US)

(73) Assignee: THE REGENTS OF THE UNIVERSITY OF CALIFORNIA, Oakland, CA (US)

(21) Appl. No.: 18/560,106

(22) PCT Filed: May 5, 2022
(86) PCT No.: PCT/US2022/027927

§ 371 Date: Nov. 10, 2023

Related U.S. Application Data
(60) Provisional application No. 63/186,713, filed on May 10, 2021.

(51) Int. Cl. G01N 15/149; G01N 15/14

(52) U.S. Cl. CPC G01N 15/149; G01N 15/1459

(57) ABSTRACT

A method of analyzing shaped particles using a flow cytometer or a fluorescence activated cell sorter (FACS) includes flowing a population of shaped particles with at least some of the population of shaped particles having cells loaded therein through the flow cytometer or FACS and optically interrogating the shaped particles in the flow cytometer or FACS to measure scattered light for each shaped particle. A target shaped particle having a cell loaded therein is detected based at least in part on a measurement of forward scattered light, side scattered light, or back scattered light. The target shaped particle may also be identified with a measured fluorescence signal level. Sorting of target shaped particles may be optimized by adjusting one or more of a drop delay or a sorting mask configuration for the flow cytometer or FACS.

### RELATED APPLICATION

This application claims priority to U.S. Provisional Patent Application No. 63/186,713 filed on May 10, 2021, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Number GM126414, awarded by National Institutes of Health. The government has certain rights in the invention.

### TECHNICAL FIELD

The technical field generally relates to methods of analyzing shaped particles or nanovials that contain cells using flow cytometers or fluorescence activated cell sorters (FACS). More particularly, the technical field relates to optimization of operational parameters in commercially available FACS instruments for use with the shaped particles.

### BACKGROUND

The ability to precisely manipulate and partition individual cells within miniaturized fluid volumes has expanded biological discovery to encompass the heterogeneity across cell populations. Traditional workflows focused on measuring bulk properties of interest from populations of cells have given way to novel microfluidic technologies enabling the parallelized assessment of these same features from each cell in a population simultaneously. Cursory insights gleaned from population averages are now being refined with a tremendous amount of single cell multi-omics data, fostering nuanced understandings of phenotypic heterogeneity and population dynamics. Early-stage adoption of these novel single cell functional screens have already proved critical across all stages of modern drug development, from antibody discovery to cell line development. Unfortunately, even with the technical progress that has been achieved, single-cell screening capabilities are often limited in scale and restricted to researchers who have the capability to implement complex microfluidic tools or have access to a few high-priced commercial platforms.

Researchers have developed techniques to address some of the challenges related to access and throughput by leveraging common flow cytometers for downstream analysis and sorting, instead of specialized instruments. For example, hybrid techniques using microfluidics to encapsulate cells within hydrogel particles, double emulsions, or hollow particle shells have been developed to create small single-cell containers that can be analyzed and sorted with standard fluorescent activated cell sorters (FACS). Still, widespread adoption of these approaches is limited due to the significant expertise and specialized equipment required for the upstream formation of compartments using microfluidic devices, and the general incompatibility of standard laboratory operations such as washing and reagent exchange once compartments have been formed. Similarly, continued miniaturization of standard micro-titer plates, have significantly enhanced the ability to screen individual cells in parallel. However, the advantages gained from enabling direct scale down of common assay protocols utilized in larger micro-titer plate formats are offset by the need to use liquid handlers and other automated robotics to accurately interface with the miniaturized well designs, significantly increasing assay costs and limiting throughputs. To unlock the potential of single cell screening workflows, new technologies must be designed that are standardized to work fully with existing infrastructure and common laboratory operations.

“Lab on a particle” technologies can enable users to perform the same fundamental operations as in microtiter plates, but at orders of magnitude higher throughputs and at volumes on the scale of individual cells. One such platform makes use of shaped hydrogel particles or “nanovials” with a void or cavity in which cells can be situated. The hydrogel shaped particle technology acts as a suspendable microcontainer that can be functionalized with chemical moieties to promote cell binding and growth. Fluids are easily exchanged around the shaped particles by simple pipetting and centrifugation steps enabling exposure to different chemical stimuli or staining reagents. The cavity of the shaped particles can be sealed in parallel through emulsification with biocompatible oils preventing molecular cross-talk between compartments. Like a well-plate bottom, the hydrogel surfaces of shaped particles can act as a substrate for performing biomolecular assays such as sandwich immuno-assays. Importantly the emulsification process is reversible allowing for viable recovery of cells, staining, and analysis or sorting using FACS. Because traditional FACS systems were designed to analyze and sort cells such as leukocytes, and the shaped particles are larger and possess a unique morphology when compared to typical mammalian cells, it is important to innovate when creating new workflows and discover operational parameters that enable new types of analysis and sorting of the shaped particles across a range of different makes and manufacturers of FACS instruments.

### SUMMARY

Three different commercially available FACS instruments were used to perform a comprehensive characterization of the conditions suitable to analyze and sort shaped particles. In particular, the following were characterized: (1) the limit of detection (LOD) and dynamic range for measuring fluorescently-labeled protein binding to the shaped particles, (2) the sorting settings that led to optimal purity and efficiency of sorting, and (3) the ability to uniquely identify and enrich cell-containing shaped particles using scatter signal. To understand the limit of detection and dynamic range of affinity assays on shaped particles a dilution sweep was performed using fluorescent streptavidin with biotinylated shaped particles and compared with microscopy measurements. Using FACS, it was found that the shaped particles could bind ˜10⁸ fluorescent molecules and had a LOD down to 10⁴ molecules, an order of magnitude improved over fluorescence microscopy measurements with a cooled CMOS camera. For each instrument the sorting parameters were systematically adjusted such as drop delay, sort masks, and sample buffers to identify the optimal conditions for sorting different size ranges of shaped particles. Conditions were identified that enabled sorting purities as high as 99% and sort recovery rates of >90%. The shaped particles or nanovials are unique in that they are transparent and scatter only weakly due to their hydrogel composition and small index of refraction difference compared with surrounding medium. By exploiting this property, it has been demonstrated that a scatter signal alone can be used to identify, gate, and sort cell-containing shaped particles from a background of empty shaped particles and free cells. The ability to enrich these populations with purities >90% was further demonstrated.

In one embodiment, a method of analyzing shaped particles using a flow cytometer or a fluorescence activated cell sorter (FACS) includes: providing a population of shaped particles with at least some of the population of shaped particles having cells loaded therein. The population of shaped particles is then flowed through the flow cytometer or FACS and are optically interrogated to measure scattered light for each shaped particle. Targeted shaped particles having cells loaded therein are identified based at least in part on a measurement of forward scattered light, side scattered light, or back scattered light.

The method may further include sorting the targeted shaped particle(s) having a cell loaded therein based on the measurement of forward scattered light being above a threshold level and the measurement of side scattered light or back scattered light being above a threshold level. The sorting may also include encapsulating the targeted shaped particle(s) within a droplet in a stream of droplets and deflecting the droplet with the shaped particle contained therein. In some embodiments, at least two adjacent droplets from the stream of droplets are also sorted with the droplet that contains the target particle.

In another embodiment, a method of sorting shaped particles using a flow cytometer or a fluorescence activated cell sorter (FACS) includes flowing a population of shaped particles through the flow cytometer or FACS and optically interrogating the shaped particles to measure one or more of: a forward scatter signal, a side scatter signal, a fluorescence signal for each shaped particle. The flow cytometer or FACS adjusts one or more of a drop delay or a sorting mask configuration for the flow cytometer or FACS and the shaped particles are sorted based at least in part on the measured forward scatter signal, side scatter signal, or fluorescence signal.

In another embodiment, a method of detecting the presence of molecules using shaped particles includes: providing a population of shaped particles with at least some of the population of shaped particles having the molecules contained therein or bound thereto. The population of shaped particles is flowed through a flow cytometer or fluorescence activated cell sorter (FACS). Shaped particles that have the molecules contained therein or bound thereto are identified based on a fluorescence signal emitted from the shaped particles, wherein the signal is detected at or below a threshold level of molecules per shaped particle. In one embodiment, the threshold limit is at or below 10⁵ molecules/shaped particle. In another embodiment, the threshold limit is at or below 10⁴ molecules/shaped particle.

### BRIEF DESCRIPTION OF THE DRAWINGS

FIG. 1A schematically illustrates a shaped particle that contains a cell therein.

FIG. 1B schematically illustrates the input sample and output sample after scatter-based FACS sorting to isolate shaped particles containing cells. A mixture of free cells, empty shaped particles, and cell-containing shaped particles are analyzed to identify unique scatter signatures and/or fluorescence signal(s) for each population and then sorted to enrich shaped particles that contain cells therein.

FIG. 2A schematically illustrates the shaped particles containing sub-nanoliter volume cavities for single cell analysis and sorting. In (i) cavity-containing hydrogel shaped particles (nanovials) can be modified with various binding moieties to directly facilitate the adhesion and growth of loaded cells. In (ii) reagents can be easily exchanged around cells contained in the cavities or voids of the shaped particles using simple pipetting and centrifugation steps. Operation (iii) shows how the shaped particles can be sealed to create uniform sub-nanoliter compartments by emulsifying with biocompatible oil and surfactants using a pipette or through vortexing. The shaped particles can be un-sealed to perform downstream processes using a destabilizing agent to coalesce emulsions. Operation (iv) illustrates how the surface of the shaped particles can be used for capturing molecules of interest such as secreted proteins from individual captured cells. In (v), shaped particles can be sized and made from materials to be compatible with commercial flow cytometers and FACS machines for high-throughput analysis and sorting.

FIG. 2B illustrates an example flow cytometry scatter plot (top) and corresponding microscopy image (bottom) obtained after enrichment of cell-containing shaped particles using FACS.

FIGS. 3A-3C: Limit of detection (LOD) and dynamic range for detecting nanovials using fluorescence microscopy and flow cytometry. FIG. 3A: biotinylated 35 μm shaped particles were labeled with Alexa Fluor™ 488 Streptavidin (AF 488 SA—fluorescent reporter) across a 4-log range of molecules per particle. Example brightfield and corresponding fluorescence images are shown over the range of conditions. Fluorescence images of particles with the same LUT values are shown in the middle column and images with LUTs maximized for contrast are shown to the right. FIG. 3B: the fluorescently-labeled shaped particles were analyzed using fluorescence microscopy and three flow cytometers (BD FACSAria™ II, SONY SH800, On-chip Sort). Intensity distributions are shown for the Sony SH800 and fluorescence microscopy. Inset graphs show a zoomed in view of lower concentrations of streptavidin for microscopy and show higher gain/voltage settings for the flow cytometers. FIG. 3C: the mean intensity for fluorescence microscopy and the intensity for the various flow cytometers are plotted against the number of molecules per particle on a log-log plot. The vertical dashed line indicates the limit of detection (LOD) for each instrument and the arrow indicates the linear portion of the dynamic range. For microscopy measurements n>200 shaped particles were measured for each condition. For flow cytometry measurements n>5000 events were measured.

FIGS. 4A-4D: Optimized Nanovial sorting parameters on the FACSAria™ II. FIG. 4A: schematic showing an overview of the FACSAria™ FACS system and important parameters that can be adjusted to fine tune sorting. The drop delay defines the expected time between the interrogation event and the formation of the aerosol droplets. Sorting masks adjust the stringency by which an event will be sorted based on its predicted positioning. FIG. 4B: Single nanovials are gated from the forward scatter area (FSC-A) and side scatter area (SSC-A) density scatter plots using the FACSAria™ II system (circled region). The arrow points to a fluorescence density scatter plot of the nanovials in two fluorescence channels. 17.7% of nanovials were target nanovials with high AF488 streptavidin (AF488 SA) fluorescence. After sorting these AF488 fluorescing nanovials are accumulated with high purity (inset). FIG. 4C: it was found that using a 130 μm nozzle and manually adjusting the drop delay at a different value from the calibrated baseline resulted in improved sort recovery efficiency. For each condition 100 events were sorted into different wells of a 96-well plate and performance was quantified by counting recovered events. It was found that over a 5-fold improvement in recovery was achieved by adjusting the delay settings for 35 micron shaped particles. Improvement was also shown for 55 micron shaped particles by adjusting drop delay manually away from the baseline value. FIG. 4D: various sort masks were tested and evaluated based on both purity of the sort and the sort recovery efficiency. Fluorescently labeled shaped particles were spiked into a nonlabelled population at a ratio of 1:10, sorted and then characterized using fluorescence microscopy. The optimal settings for purity are Phase 16 and Purity 24 and sort recovery efficiency are Phase 16 and Purity 16.

FIGS. 5A-5D: Optimized shaped particle sorting parameters on the SONY SH800. FIG. 5A: illustrates an overview schematic of the SONY SH800 system and sorting definitions. Sorting decisions are typically made in reference to the relative position of the target event within a droplet and the distance between target and off-target events. FIG. 5B: an overview of four common sorting modes on the SONY SH800 and the decision-making breakdown for the 130 μm chip. FIG. 5C: Single nanovials are gated from the forward scatter area (FSC-A) and side scatter area (SSC-A) density scatter plots using the Sony SH800S system (circled region). To the right is a fluorescence density scatter plot of the nanovials in two fluorescence channels. 18.2% of nanovials were target nanovials with high AF488 streptavidin (AF488 SA) fluorescence. After sorting these AF488 fluorescing nanovials are accumulated with high purity (inset). FIG. 5D: the different sort modes were tested and evaluated based on both purity of the sort and the sort recovery efficiency. Fluorescently-labeled shaped particles were spiked into a non-labeled population at a ratio of 1:10, sorted and then characterized using fluorescence microscopy. The optimal settings for purity and sort recovery efficiency was the Single Cell mode.

FIGS. 6A-6D: Optimized shaped sorting parameters on the On-chip Sort. FIG. 6A: scatter plots of side scatter height (SSC(H)) and fluorescence height used to identify stained shaped particles. FIG. 6B: size dependent sorting up to 85 μm nanovials. FIG. 6C: density matching to increase uniformity of sort event rates and improve performance. FIG. 6D: purity and recovery efficiency for different buffers and pressure settings.

FIG. 7A: schematically illustrates a sample holder containing free cells, empty shaped particles or nanovials, and shaped particles or nanovials loaded with cells.

FIG. 7B: scatter plot showing forward scatter area (FSC-A) versus side scatter area (SSC-A, also called back scatter area) plot of a sample containing shaped particles, cells, and shaped particles containing cells.

FIG. 7C scatter plot showing the shaped particles and cells were stained with unique fluorescent labels (Cell Tracker Deep Red and Alexa Fluor 488 (Streptavidin)) in order to accurately discriminate each population.

FIG. 7D illustrates a scatter plot showing forward scatter area (FSC-A) versus side scatter area (SSC-A) that is gated to show regions that contain empty shaped particles/nanovials and shaped particles/nanovials with cells.

FIG. 7E illustrates a fluorescence microscope image of shaped particles/nanovials with cells gated and sorted based on fluorescence parameters. Gate is from upper right quadrant of FIG. 7C.

FIG. 7F illustrates a brightfield microscope image of the shaped particles/nanovials with cells gated and sorted using scatter parameters. Gate is from “Nanovials with cells” gate in FIG. 7D.

FIG. 7G: samples were sorted based on fluorescent gating alone and scatter gating alone and imaged to compare enrichment of the cell-containing shaped particles.

### DETAILED DESCRIPTION OF ILLUSTRATED EMBODIMENTS

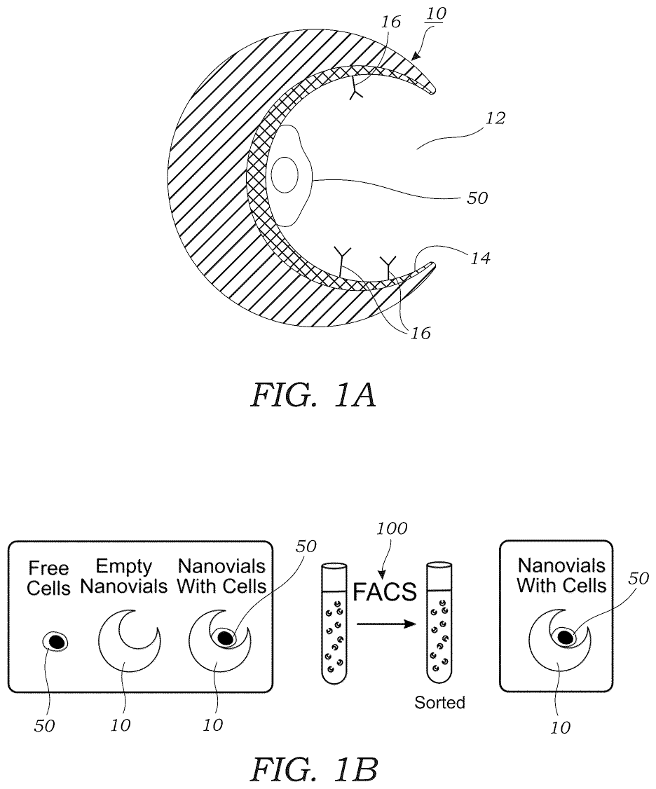

As seen in FIG. 1A, the shaped particles 10 (sometimes also referred to as nanovials) typically are micrometer sized particles (e.g., three-dimensional shaped particles). Generally, the shaped particles 10 have a longest dimensional length of around 100 μm or less. For applications that require the loading of cells 50 into/onto the shaped particles 10, the shaped particles 10 typically have a minimum dimensional length of at least 10 μm. Of course, in other applications, there is no lower limit on the size of the shaped particles 10. In embodiments in which flow cytometers or fluorescence activated cell sorters are used to analyze or sort shaped particles 10, the shaped particles 10 are preferably between ˜30 μm and ˜60 μm in a maximum dimension. The shaped particles 10 may be formed from biocompatible materials or polymers. In one embodiment, the shaped particles 10 are formed from polyethylene glycol (PEG).

The shaped particle 10 includes a void or cavity 12 as seen in FIG. 1A. The void or cavity 12 may be open to the external environment of the shaped particle 10 as illustrated in FIG. 1A. Alternatively, the void or cavity 12 may be completely enclosed by a surface of the shaped particle 10 (e.g., spherical particle with hollow void or cavity 12 therein). In still yet another embodiment, the shaped particle 10 may also include a particle (e.g., spherical particle) with a number of separate voids or cavities 12 distributed within the shaped particle 10. The shaped particles 10 may have a number of shapes including: crescent shaped, bowl shaped, moon shaped, capsule shaped, concentric sphere shaped. The shaped particles 10 may have a void or cavity 12 sized to fit a single cell 50 or, in some embodiments, a plurality of cells 50. For example, the shaped particles 10 may have a void or cavity 12 with a longest dimension of 10 μm-30 μm. In a preferred embodiment, the void or cavity 12 (or collection of multiple voids or cavities 12) is dimensioned to hold a sub-nanoliter volume of fluid. The fluid may include an aqueous-based fluid. As explained herein, in some preferred embodiments, the shaped particles 10 preferably are designed to carry or hold cells 50 within the void or cavity 12. The cell(s) 50 may be located within the volume of fluid within the void or cavity 12. In some embodiments, the cell(s) may adhere or become adherent to a surface of the shaped particle 10 within the void or cavity 12. Of course, in other embodiments, the shaped particles 10 may not hold cells 50. The shaped particles 10 may hold molecules or other substances with or without cells 50.

In one embodiment, the shaped particles 10 have a localized cell adhesive region 14. The localized cell adhesive region 14 is preferably located along the inner surface of the void or cavity 12. The localized cell adhesive region 14 may be functionalized with biotin and/or streptavidin to enable binding directly to biotin or streptavidin labeled cells. Alternatively, or in addition, the localized cell adhesive region 14 comprises cell adhesive or binding moieties specific to other cell surface markers or labels such as antibodies, aptamers, nucleic acids, oligonucleotides, extracellular matrix proteins or fragments thereof, and the like. The shaped particles 10 and/or the localized cell adhesive region 14 may be functionalized with one or more affinity capture agents 16. The affinity capture agent 16 may include, for example, biotin, streptavidin, a capture antibody or fragment thereof, enzyme, protein, protein fragment, nucleic acid, aptamer, or the like. The affinity capture agent 16 may be secured to the localized cell adhesive region 14 or the shaped particle 10 using a linker molecule such as, for example, biotin and/or streptavidin. By populating the localized cell adhesive region 14 with affinity capture agents 16, the shaped particles 10 can be used to locally enrich secreted biomolecules or other products secreted or released from cells 50. In some embodiments, the shaped particle 10 comprises at least 10⁸ affinity capture agents 16. In some embodiments, the shaped particle 10 comprises at least 10⁶ affinity capture agents 16. In addition, the localized cell adhesive region 14 populated with the affinity capture agents 16 advantageously reduces unwanted leakage or crosstalk of secreted or released biomolecules interacting with other shaped particles 10. The localized cell adhesive region 14 populated with the affinity capture agent 16 also enables single cell secretion assays to be performed without the need for the formation of shaped particle 10 emulsions (i.e., no need for formation of dropicles in which shaped particles 10 are surrounded by an oil phase) although such emulsions may still be used in some embodiments.

Shaped particles 10 containing cells 50 and secreted or released biomolecules captured on affinity capture agents 16 may be labeled with fluorescent reporters to characterize the amount, affinity, specificity, or other properties of secreted or released biomolecules. Fluorescent reporters may include fluorescently labeled antibodies, fluorescently-labeled antigens, fluorescently-labeled oligonucleotides, fluorescently-labeled ligands or proteins, enzymes that generate localized fluorescent signal, and the like. Fluorescent reporters (e.g., antigens) may be exposed to shaped particles 10 with captured biomolecules (e.g., antibodies) at different concentrations to probe the affinity of the captured biomolecules to the fluorescent reporter.

The shaped particles or nanovials 10 can then be analyzed and/or sorted through a flow cytometer or FACS instrument 100 as seen in FIGS. 1B, 2A, 4A, 5A at high rates. For example, depending on the size of the shaped particle 10 the event rates for analysis or sorting may be >100 events/sec, >400 events per second, or up to ˜1000 events/sec. The flow cytometer or FACS instrument 100 optically interrogates the shaped particles 10 that flow through the device (typically with laser interrogation). Detectors that are part of the flow cytometer or FACS instrument 100 are used to detect or measure one or more optical signals emitted by the shaped particles 10. The detected or measured optical signals include, for example, fluorescence intensity. In addition, or separate from fluorescence detection, scatter measurements may be performed by the flow cytometer and/or FACS instrument. This includes forward scatter, side scatter, and back scatter measurements. These measurements can be used to detect or sort the shaped particles 10. For example, in one embodiment, one or more target shaped particles 10 having respective cells 50 loaded therein are detected based on at least in part a measurement of forward scattered light, side scattered light, or back scattered light (or combinations thereof). It should be appreciated that in other embodiments, cells 50 are not located in the target shaped particles 10 but rather molecules. The number or amount of molecules present in the shaped particles 10 may be determined based, for example, on the measured optical signal (e.g., fluorescence intensity level).

The target shaped particles 10 may be sorted based on the detected/measured optical signals. For example, in one example, the one or more target shaped particles 10 having respective cells 50 (or in another embodiment, molecules) loaded therein are sorted based on the measurement of forward scattered light being above a threshold level and the measurement of side scattered light or back scattered light being above a threshold level. Of course, sorting may also happen based on the measurement of forward scattered light being below a threshold value and the measurement of side scattered light or back scattered light being below a threshold value (e.g., non-targeted particles are gated and sorted out). This is an example of sorting based on gating of two parameters (e.g., threshold of side scatter light or back scattered light). Sorting of shaped particles 10 may also occur based on measured fluorescence signal level. Combinations of fluorescence signal level and scattered light (forward, side, and/or back) may also be used to gate or sort shaped particles 10. In other embodiments, the target shaped particle is detected (and optionally sorted) based on a gating of two or more of measured forward scattered light, measured side scattered light, or measured back scattered light. It should be appreciated that gating is not necessarily limited to thresholds being above or below a particular measured level. For example, various axes may be plotted against one another (e.g., a typical flow cytometer or FACS plot) and certain regions of datapoints may be circumscribed by lines or boundaries that define the particular gating criteria. Typical flow cytometer and FACS instruments 100, for example, allow the user to create these gating criteria manually using an input device like a mouse, pen, or the like to select the gated region.

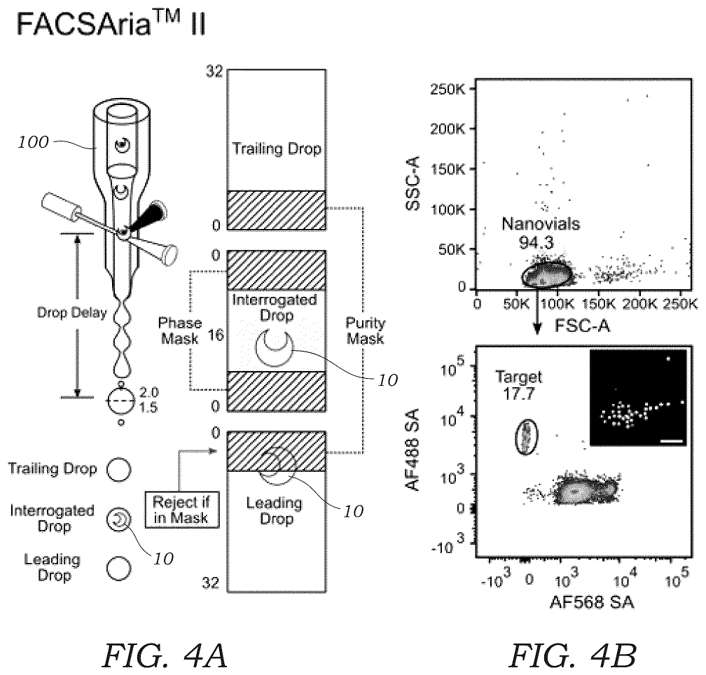

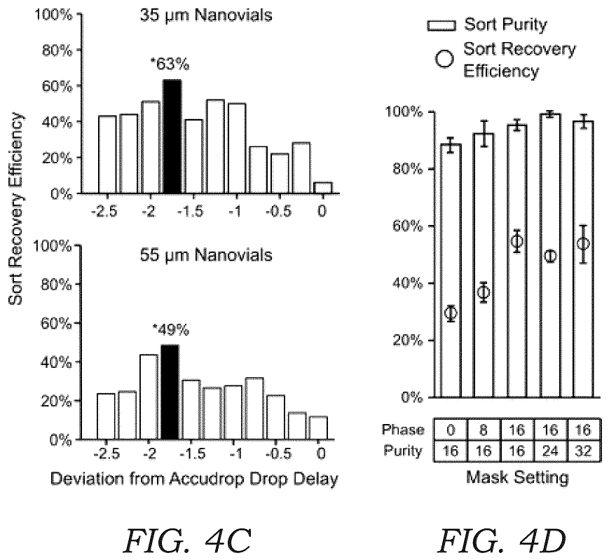

Various operational parameters of the particular flow cytometer or FACS instrument 100 may be adjusted to optimize purity or sort recovery efficiency. These include, by way of example, adjusting the drop delay and using different sorting mask configurations. The drop delay is the amount of time between the laser interrogation point and the droplet break-off point. The drop delay determines how long the flow cytometer or FACS instrument 100 waits before it applies a charge for sorting a target shaped particle 10. The drop delay is generally measured as droplet periods which is 1/droplet frequency. The FACS instrument may also include on-chip instruments which pass shaped particles 10 through microfluidic flow channels for sorting instead of aerosolized droplets. In such instruments, density-modulating agents may be used to alter or tune the density of the solution containing the shaped particles 10 to improve the sorting rate and prevent settling and clogging. For example, FIG. 4C illustrates an example of how drop delay was adjusted (manually) in the FACSAria™ II instrument 100 to improve sort recovery efficiency. It was found that over a 5-fold improvement in recovery was achieved by adjusting the delay settings for 35 micron shaped particles 10.

Mask configurations may vary depending on the type of FACS instrument 100. For example, the FACSAria™ II instrument 100 has three different types of mask settings. These include a yield mask, purity mask, and phase masks. The yield mask setting defines how close to the edge of the drop (in 1/32 drop increments), a particle of interest can be located before sorting an additional drop. The purity mask setting defines how close (in 1/32 drop increments), a contaminating drop can be located before ignoring the drop being interrogated. The phase mask restricts drop deflection when an event is too close to the edge of a drop or when there are events close to the edge of adjacent drops. The phase mask is used to improve counting accuracy and side-stream quality at the expense of yield. Multiple masks can be combined in a so-called sort precision mode. Exemplary default sort precision modes include purity, yield, single cell, normal, and fine tune. New precision modes may be customized for different sorting configurations. FIG. 4A illustrates an example of a purity mask that looks at the leading droplet and trailing droplet. A phase mask, however, is applied to the interrogated droplet (FIG. 4A). FIG. 4D illustrates an example how different phase/purity mask settings are used to optimize for purity (16/24) and sort recovery efficiency (16/16) for shaped particles 10.

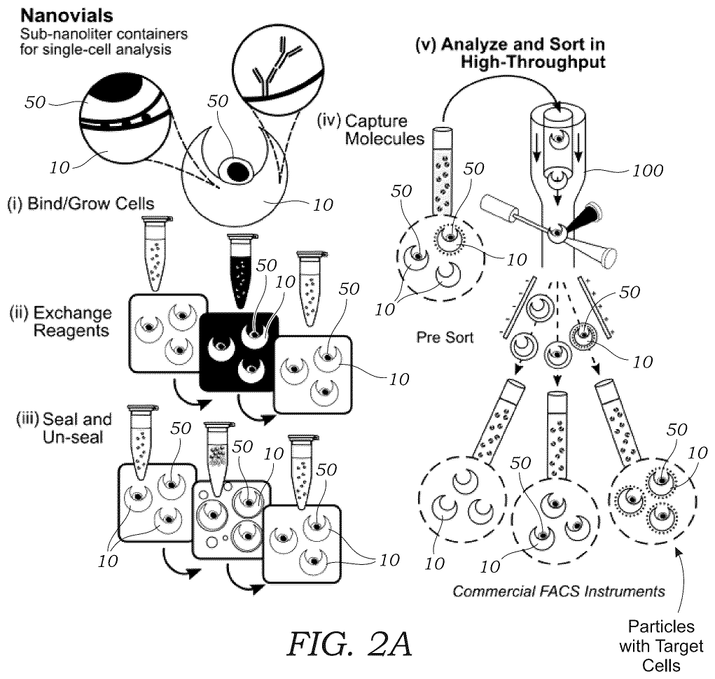

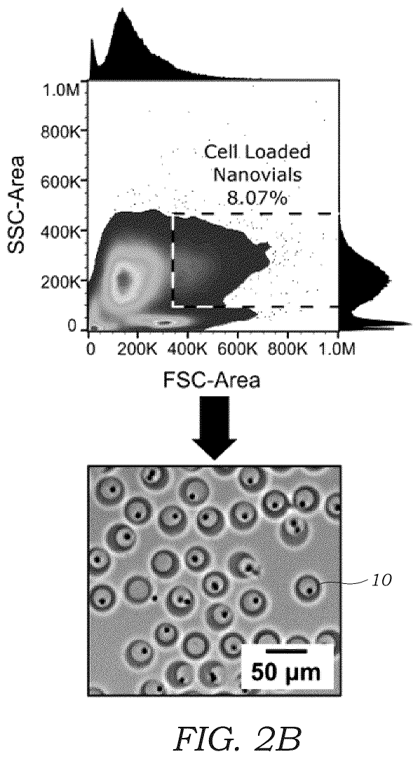

With reference to FIGS. 2A and 2B, an exemplary sequence of operations or workflow; is disclosed for using shaped particles 10 for single cell analysis and sorting. In operation (i) of FIG. 2A, cavity-containing hydrogel shaped particles 10 (nanovials) can be modified with various binding moieties to directly facilitate the adhesion and growth of loaded cells 50. In operation (ii) reagents can be easily exchanged around cells 50 contained in the cavities or voids 12 of the shaped particles 10 using simple pipetting and centrifugation steps. Operation (iii) shows how the shaped particles 10 can be sealed to create uniform sub-nanoliter compartments by optional emulsifying with biocompatible oil and surfactants using a pipette or through vortexing. The shaped particles 10 can be un-sealed to perform downstream processes using a destabilizing agent to coalesce emulsions. Operation (iv) illustrates how the surface of the shaped particles 10 can be used for capturing molecules of interest such as secreted proteins. In (v), shaped particles 10 can be sized to be compatible with commercial flow cytometers and FACS machines for high-throughput analysis and sorting. FIG. 2A illustrates how target cells 50 loaded into sharped particles 10 are sorted in to an enriched population that can be collected for further analysis and/or growth. FIG. 2B illustrates an example flow cytometry scatter plot (top) and corresponding microscopy image (bottom) obtained after enrichment of cell-containing shaped particles 10 using FACS. A gating region can be defined using side scattering and forward scattering parameters to identify those shaped particles 10 that contain loaded cells 50.

### Experimental

In the studies herein, the use of shaped particles 10 with three separate commercially available fluorescence activated cell sorter instruments 100 were characterized: (1) BD FACSAria™ II (BD Biosciences), (2) SONY SH800 (Sony Biotechnology), and (3) On-chip Sort (On-chip Biotechnologies). Each instrument's ability to detect fluorescence signal on shaped particles 10 was compared with fluorescence microscopy, parameters to maximize the purity and efficiency of shaped particle sorting were identified, and approaches to analyze or sort cell-loaded shaped particles 10 using scatter measurements alone were developed. By identifying the optimal instrument parameters and trade-offs between each system one is able to choose the appropriate instrumentation to meet experimental or other research needs.

### Improved Dynamic Range and Limit of Detection Using FACS Compared to Microscopy

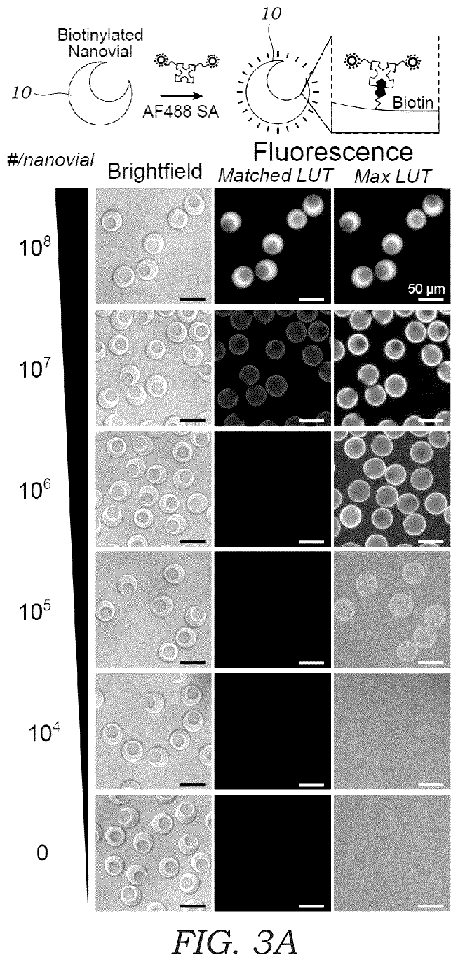

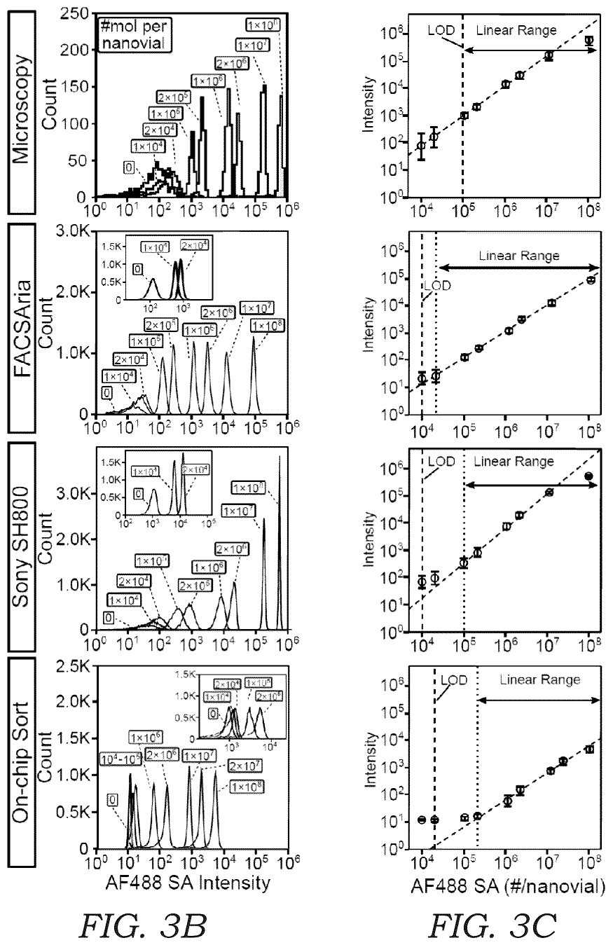

It was found that all three cell sorter instruments 100 that were evaluated showed improved limit of detection and dynamic range in detecting fluorescently-labeled shaped particles 10 compared to microscopy. A unique feature of the shaped particles 10 is the ability to bind biomolecules (e.g., cell secretions) to affinity reagents 16 attached to the hydrogel matrix and detect their presence using labels, such as fluorescent labels. Such secretion assays and methods of producing shaped particles 10 for such secretion assays are disclosed in International Patent Publication No. WO2020037214A1, which is incorporated herein by reference. A core advantage of flow cytometers is their ability to measure fluorescent signal associated with 100,000's to 10's of millions of unique events, such as microbeads and cells, easily and with limits of detection approaching hundreds of fluorophores. The ability to detect fluorescent protein bound to shaped particles 10 was assessed using flow cytometry mimicking the signal from a biomolecular assay on a shaped particle 10 (e.g., single cell secretion assay). Fluorescent streptavidin was bound to biotinylated shaped particles 10 across a 4-log range of concentrations to characterize both the limit of detection and the dynamic range of the instruments as seen in results presented in FIGS. 3A-3C. Fluorescence microscopy measurements were taken in parallel as a baseline reference as seen in FIG. 3A.

Using fluorescence microscopy, it was found that signal could be detected on the shaped particles 10, above background noise, starting at around 100,000 molecules per particle. The fluorescence intensity of each shaped particle 10 was determined utilizing an automated image analysis algorithm to detect each shaped particle 10 in the image and integrate the fluorescence intensity across the shaped particle 10. Localized normalization in the automated analysis algorithm was required to reach this performance level by reducing variation due to illumination non-uniformity across the images. Fluorescence images reveal that the streptavidin first binds to the outer surface of the shaped particles 10 at lower concentrations and then can transport and react further into the hydrogel matrix of the shaped particles 10 as biotin groups become saturated. It was found that biotin groups were saturated at ˜10⁸ molecules per shaped particle 10 for the fabrication conditions used.

It was found that measurements with the flow cytometers 100 improved the limit of detection by an order of magnitude (LOD <10,000 molecules/particle) when compared to fluorescence microscopy. Likewise, the dynamic range and the linear range of the measurements were improved by an order of magnitude (5 log and 4 log respectively). Shaped particles 10 labeled with <10⁵ molecules were initially difficult to distinguish from non-labeled controls. However, through adjustment of the PMT voltage or gain parameters lower signal samples could be clearly resolved (FIGS. 3B-3C). This suggests that optimal parameters should be selected based on experiment need, with lower PMT voltage favoring increased resolution and higher values providing greater dynamic range. All FACS systems performed better then microscopy. Both the SONY SH800 and On-chip Sort have disposable polymer chips in the optical path which may slightly reduce the optical performance compared to the quartz cuvette using the BD FACS Aria II instrument.

### Sorting Nanovials Using FACS

Optimal settings and conditions were identified for sorting shaped particles 10 of different sizes by systematically adjusting sorting settings on the different FACS instruments 100. Each instrument 100 has unique settings and parameters that can be adjusted so the characterization of each system is discussed separately.

### FACSAria™ II

To test the sorting performance of the FACSAria™ II instrument 100 a population of fluorescently-labeled shaped particles 10 were spiked into a population of shaped particles 10 (i.e., nanoparticles) with a second fluorescent label at a ratio of 1:10 and sorted into a 96-well plate using its index sorting feature. Samples were then imaged using fluorescence microscopy to assess both the purity of the sort and the sort recovery efficiency as defined by:

$$\begin{matrix}Sort Recovery Purity = \frac{n umber of target nanovials in well}{n umber of total nanovials in well} \times 100 \% \\ Sort Recovery Efficiency = \frac{n umber of target nanovials in well}{n umber of reported sorts} \times 100 \%\end{matrix}$$

The Aria system utilizes an integrated Accudrop system to calibrate the drop delay between the detected event and the sorting point. This value can shift day to day and it is recommended to calibrate it before each experiment utilizing associated calibration beads. It was found that the sort recovery efficiency for both 35 μm and 55 μm diameter shaped particles 10 was low when using the baseline calibration value. By sweeping drop delay measurements manually in 0.25 increments it was found that an Accudrop delay value of −1.75 led to the best recovery efficiency for both shaped particle 10 sizes tested. It was hypothesized that this delay is due to a modified velocity of the shaped particles 10 in the nozzle. Shaped particles 10 cover a larger fraction of the cross section of the flow compared to a cell which leads to a change in average velocity in the non-uniform flow cross-section compared to smaller cells.

Sorting masks can be adjusted to maximize purity or sort recovery efficiency. The Aria system defines its masks in reference to the interrogated droplet (the droplet where the target shaped particle 10 is predicted to be), the leading droplet (the droplet in front of the target), and the trailing droplet (that follows the target droplet). Each droplet is broken up into 32 fractions that define the relative coverage of each mask. The purity mask defines the fraction of the leading and trailing droplet in which the presence of another event will abort the sort. The phase mask defines which region of the interrogated droplet the target should be predicted to be in to proceed with a sort. Using a similar strategy as with the drop delay, these conditions were swept and quantified the sort purity, sort recovery efficiency, and the abort rate for each. The best purity performance was found for the most restrictive purity masks. It was further found that more restrictive phase masks improved the sort recovery. It was hypothesized that since the particles are larger than normally sorted objects, target events near the edge of each drop may disrupt the stability of the aerosolizing stream, leading to over or under-deflection during sorting and a missed collection in the target container.

### SONY SH800

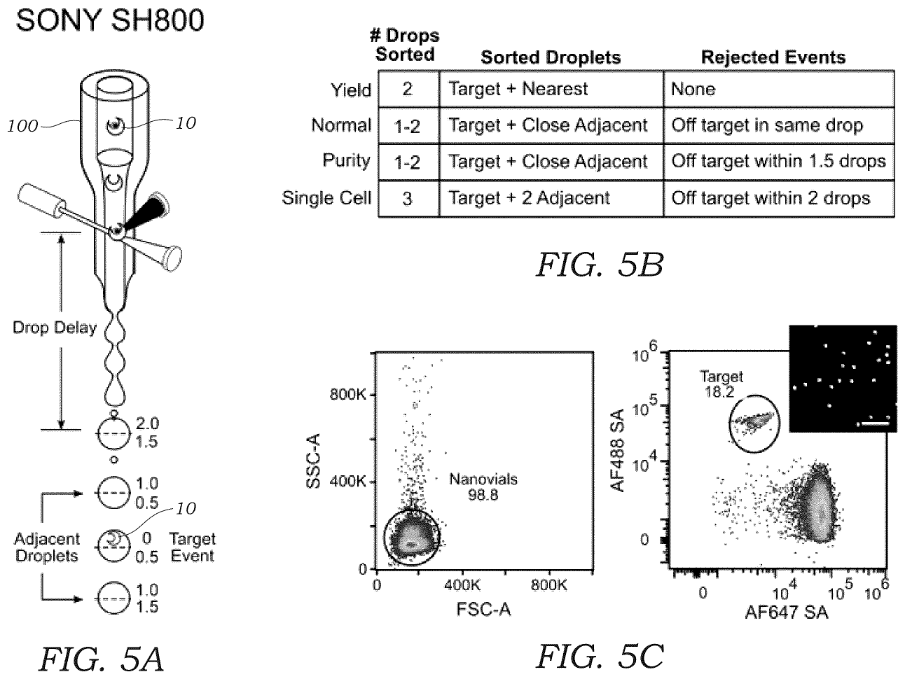

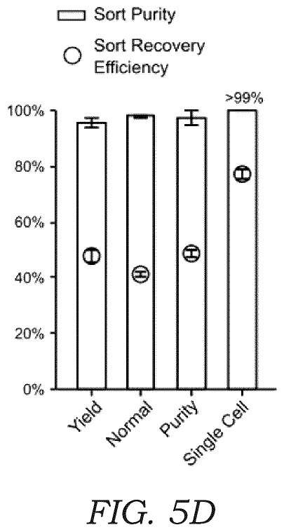

Sorting parameters were also identified for the SONY SH800 instrument 100 as described above. It was found that both 35 μm and 55 μm shaped particles 10 were compatible with the larger 130 μm chip available for the system and optimization was focused for this setup. For the SH800 sort masking takes into account the target event droplet, adjacent droplets and the relative position of the target event to off target events (FIG. 5A). The standard sorting modes are yield, normal, purity, and single cell and descriptions of these sort criteria are shown in FIG. 5B. To characterize each sorting mode, fluorescently-labeled shaped particles 10 spiked into non-labeled shaped particles 10 were sorted into a 96-well plate for each mode and sort purity and sort recovery efficiency was quantified using fluorescence microscopy. As expected, the lowest purity resulted from yield mode, as this mode does not reject a sort in the presence of adjacent off-target events. Despite this, purity was quite high and increased incrementally with the more restricted sorting modes, with the highest being for Single Cell recovery mode which had 100% purity across three (3) separate sorts (FIG. 5D). Interestingly, the Single Cell mode had significantly higher sort recovery efficiency compared to any of the other modes. This was attributed to the fact that Single Cell mode will sort the target event droplet and both of the adjacent droplets. As described above, the larger size of the shaped particles 10 can potentially cause more inconsistency in sort timing as well as affect droplet deflection accuracy. Thereby including three (3) droplets in the sort led to better recovery compared to the other modes which only sort 1-2 of the droplets depending on the event conditions. One downside to this is that Single Cell mode will only sort if there are no overlapping events in the adjacent droplets leading to a higher frequency of aborted events and reduced sample yield.

### On-Chip Sort

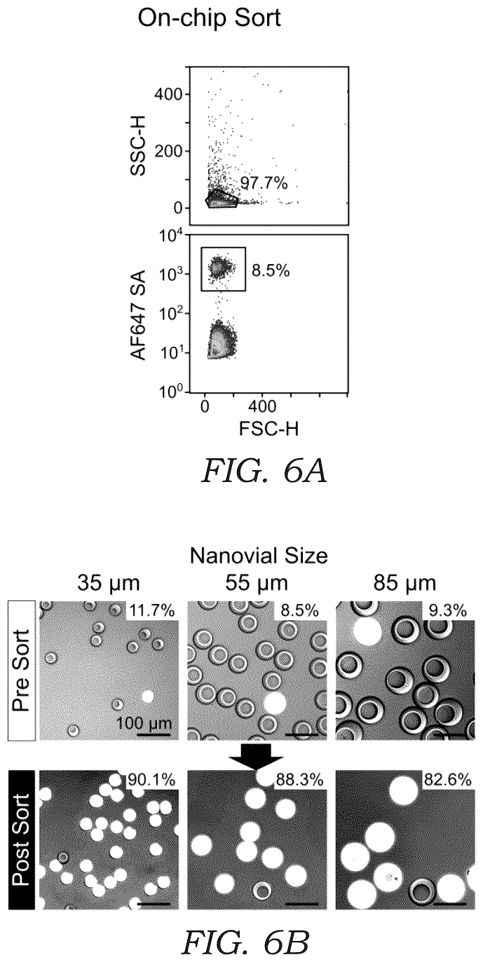

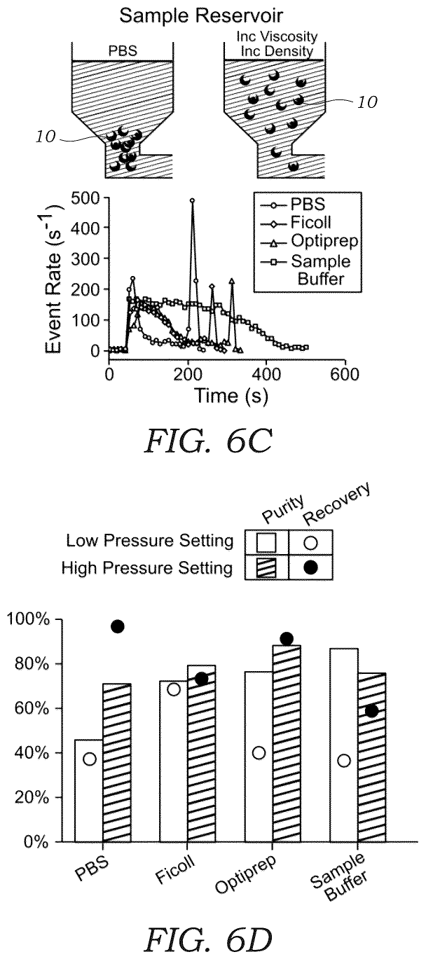

The On-chip Sort instrument 100 is unique compared to the other two systems in that it is aerosol free and has a larger range of particle size compatibilities. Like the SH800 and FACSAria™ instruments 100 a fluorescently labeled subpopulation of shaped particles 10 was spiked into a larger population of unlabeled shaped particles 10 at a 1:10 ratio and sorted to characterize purity and recovery efficiency. It was found that one could easily sort shaped particles 10 up to 85 microns in diameter with the larger 150 micron chip available for the instrument achieving ˜10-fold enrichment (FIGS. 6A-6B). The instrument 100 does not have a sample mixer and it was noted that shaped particles 10 settled and tended to aggregate at the sample inlet (FIG. 6C). This often resulted in an initial moderate event rate followed by little to no events and eventually to a large spike in events as the last of the sample fluid was pushed through the channel. To address this. Ficoll and Optiprep were tested, two well-known density-modulating agents used for cell separation processes to tune the density of the sample solution such that the shaped particles 10 are nearly neutrally buoyant. The On-chip sample buffer was also tested which aims to increase settling time through increased viscosity. Each of these experiments was conducted with 55-micron shaped particles 10 as these samples gave the most inconsistent sorting and settling results in PBS. In addition to the different solutions the On-chip instrument 100 has two standard pressure modes, low-pressure mode, optimized for lower viscosity solutions such as PBS and high-pressure mode used for sorting in more viscous solutions. It was found that in general using the higher-pressure mode as well as using the buoyancy matched and higher viscosity solutions led to much more consistent event rates and improved the purity and recovery rates (FIG. 6D). Optiprep resulted in the highest purity of the solutions, however, it tended to cause large variation in scatter signal. For further studies the Ficoll sample solution was used as it had the best balance of sorting results and optical properties. Although purity was lower for the On-chip Sort, the sort recovery efficiency and yield were high. Since no aerosols are generated, the device 100 is more forgiving with compatible shaped particle 10 sizes and there is no risk of sorted droplets missing the recovery container. During default operation, the On-chip Sort targets maximizing yield. However, because sample loading, sorting, and collection all occurs within the same self-contained chip interface higher purities can be achieved by collecting and re-sorting previously sorted samples.

### Scatter Based Enrichment of Cell-Containing Subpopulations

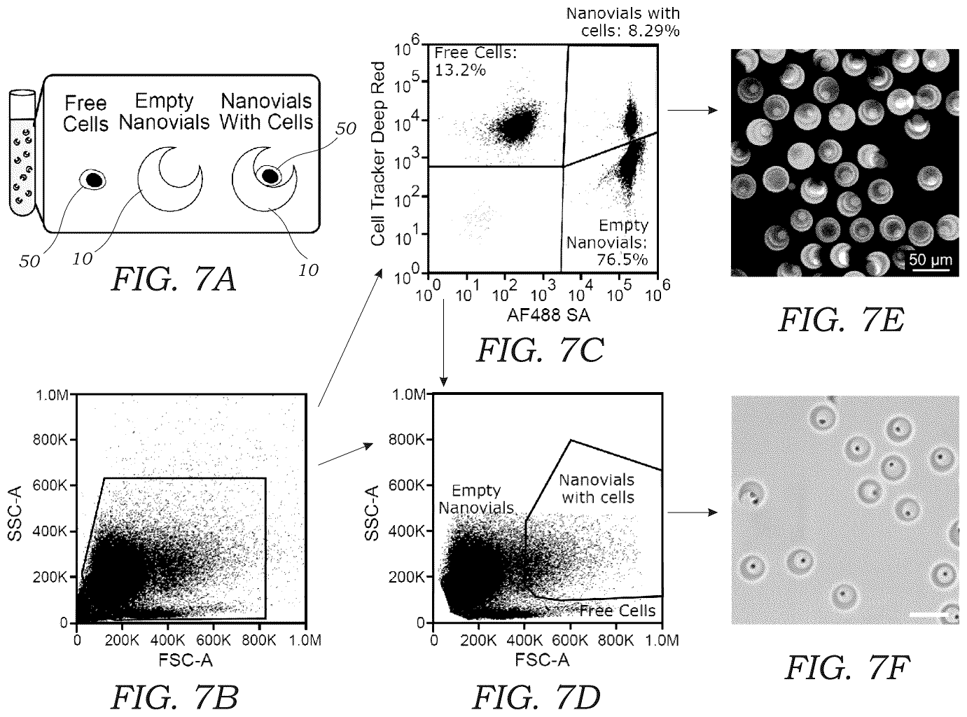

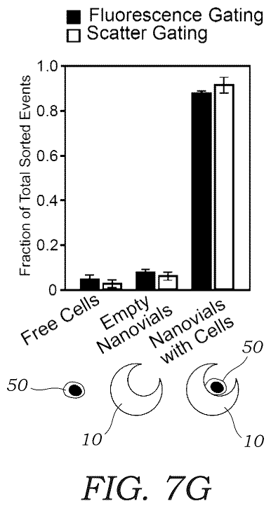

Unique scatter signatures can be used to identify a population of cell-containing shaped particles 10 among a background of free cells and shaped particles 10 without cells as seen, for example, in FIGS. 7B and 7C. Scatter based gating is powerful for both cleaning up data by removing debris or unwanted populations of cells and identifying sub-populations of interest. This can be advantageous for getting more accurate results, purer samples, and can free up fluorescent channels for additional stains. Along with traditional cell surface and intracellular stains, shaped particles 10 are powerful in that secreted or released molecules can be captured and also stained for higher levels of multiplexing and different assay formats. Here, unique scatter signatures associated with shaped particles 10 containing cells 50 were identified (FIGS. 7A-7G) in order to improve analysis capabilities and free up additional fluorescent channels for other labels on the cell 50 and/or shape particle 10.

As a model system (FIG. 7A). B lymphocytes cells 50 stained with fluorescent cell tracker were bound to fluorescently labeled shaped particles 10. A mixture of the cell-containing shaped particles 10, empty shaped particles 10, and free cells were analyzed in each of the FACS instruments 100 (FIG. 1B). Using the unique fluorescent signatures of each population it was possible to back gate each target population to their associated scatter signals (FIG. 7C). High forward scatter area (FSC-A) and back scatter area (SSC-A) parameters were generally associated with shaped particles 10 containing cells (FIG. 7D). After determining the associated population, a conservative gate was created based on scatter signal alone and sorted, aiming to enrich for shaped particles 10 containing cells (FIGS. 7D, 7F). A second sample was sorted based on the fluorescence signal (high cell and high nanovial) to act as a control for optimally expected results (FIGS. 7C, 7E). Samples were then imaged using fluorescence microscopy and analyzed to compare enrichment based on fluorescent signature and scatter signature. For the SONY SH800 it was found that using SSC-A and FSC-A one was able to accurately distinguish shaped particles 10 with attached cells 50, as accurately as fluorescence staining and gating (FIG. 7G).

Here, the analysis and sorting of hydrogel shaped particles 10 was systematically characterized with commercial FACS instruments 100. By employing the high signal to noise of FACS readouts one is able to detect protein bound to affinity agents with higher fluorescence intensity and dynamic range than high end fluorescence microscopy readouts, improving performance by an order of magnitude. The sorting performance on the different FACS instruments 100 was characterized and optimal parameters were identified that improved purity and sample recovery over baseline settings. Using more traditional FACS instruments 100 such as the FACSAria™ and SONY SH800 one is able to sort shaped particles 10 up to 55 microns in diameter. With the microfluidic based On-chip Sort system it was possible to sort shaped particles 10 up to 85 microns in diameter suitable for larger cells 50 or cell colonies. Although all systems were compatible and provide strong performance with shaped particles 10, each of the systems possessed their own advantages and trade-offs in operating on shaped particles 10 which are highlighted in Table 1 below.

TABLE 1

| | Nanovial | Working Conc. | | | | | | |
|---|---|---|---|---|---|---|---|---|
| | Size | (nanovials × | Event rate | | Recovery | LOD (# | Dynamic | Linear |
| Instrument | (μm) | 10⁶/mL) | (s⁻¹) | Purity | Efficiency | mol) | Range | Range |
| FACSAria ™ | 35 | 1-4 | 300-400 | 95-99% | | ≤10⁴ | | |
| II | 55 | 0.25-1 | 200-300 | | | | | |
| | 85 | NA | NA | NA | NA | | | |
| SONY | 35 | 1-4 | 400-600 | 95-99% | | ≤10⁴ | 4 Log | 3 Log |
| SH800 | 55 | 0.25-1 | | | | | | |
| | 85 | NA | NA | NA | NA | | | |
| On-chip Sort | 35 | 0.5-2 | 100-300 | 60-90% | | ~10⁴ | 4 Log | 3 Log |
| | 55 | 0.25-0.5 | 20-50 | 60-90% | 60-90% | | | |
| | 85 | 0.1-1 | 20-100 | 60-90% | | | | |

While embodiments of the present invention have been shown and described, various modifications may be made without departing from the scope of the present invention. The invention, therefore, should not be limited, except to the following claims, and their equivalents.

1. A method of analyzing shaped particles using a flow cytometer or a fluorescence activated cell sorter (FACS) comprising:
providing a population of shaped particles with at least some of the population of shaped particles having cells loaded therein;
flowing the population of shaped particles through the flow cytometer or FACS;
optically interrogating the shaped particles in the flow cytometer or FACS to measure scattered light for each shaped particle; and
detecting one or more target shaped particles having respective cells loaded therein based at least in part on a measurement of forward scattered light, side scattered light, or back scattered light.

2. The method of claim 1, further comprising sorting the one or more target shaped particles having respective cells loaded therein based on the measurement of forward scattered light being above a threshold level and the measurement of side scattered light or back scattered light being above a threshold level.

3. The method of claim 1, further comprising sorting the one or more target shaped particles having respective cells loaded therein, wherein sorting comprises encapsulating the one or more target shaped particles within respective droplets in a stream of droplets and deflecting the droplets with the one or more target shaped particles contained therein and at least two adjacent droplets from the stream of droplets.

4. The method of claim 1, wherein the one or more target shaped particle is detected based on a gating of two or more of measured forward scattered light, measured side scattered light, or measured back scattered light.

5. The method of claim 1, wherein the shaped particles are optically interrogated for a fluorescence signal and the one or more target shaped particles are further detected at least in part on measured fluorescence signal level and wherein the one or more target shaped particles having respective cells loaded therein are sorted based on the fluorescence signal level.

6. The method of claim 1, wherein a longest dimension of the shaped particles is between 30-60 μm.

7. The method of claim 6, wherein the shaped particles are analyzed at a rate of >400 events/second.

8. A method of sorting shaped particles using a flow cytometer or a fluorescence activated cell sorter (FACS) comprising:
providing a population of shaped particles;
flowing the population of shaped particles through the flow cytometer or FACS;
optically interrogating the shaped particles in the flow cytometer or FACS to measure one or more of: a forward scatter signal, a side scatter signal, a fluorescence signal for each shaped particle;
adjusting one or more of a drop delay or a sorting mask configuration for the flow cytometer or FACS; and
sorting the shaped particles based at least in part on the measured forward scatter signal, side scatter signal, or fluorescence signal.

9. The method of claim 8, wherein the adjusting one or more of a drop delay or a sorting mask configuration is done to maximize purity of the shaped particles.

10. The method of claim 8, wherein the adjusting one or more of a drop delay or a sorting mask configuration is done to maximize sort efficiency of the shaped particles.

11. A method of detecting the presence of molecules using shaped particles comprising:
providing a population of shaped particles with at least some of the population of shaped particles having the molecules contained therein or bound thereto;
flowing the population of shaped particles through a flow cytometer or fluorescence activated cell sorter (FACS); and
detecting shaped particles that have the molecules based on a fluorescence signal emitted from the shaped particles, wherein the signal is detected at or below 10⁵ molecules/shaped particle.

12. The method of claim 11, wherein the signal is detected at or below 10⁴ molecules/shaped particle.

13. The method of claim 11, wherein the shaped particles are loaded with one or more cells therein.

14. The method of claim 11, wherein a longest dimension of the shaped particles is between about 30 to about 60 μm.

15. The method of claim 11, wherein the shaped particles are flowed through the flow cytometer or FACS at a rate of >400 events/second.
